(19) United States
(12) Patent Application Publication
(10) Pub. No.: US 2007/0259438 A1
(43) Pub. Date: Nov. 8, 2007

(54) CHROMOIONOPHORE AND METHOD OF DETERMINING CALCIUM IONS

(75) Inventors: Huarui HE, Alpharetta, GA (US); Chao LIN, Alpharetta, GA (US)

Correspondence Address: FOLEY AND LARDNER LLP;SUITE 500, 3000 K STREET NW, WASHINGTON, DC, 20007, US

(73) Assignee: OPTI Medical Systems, Inc.

(21) Appl. No.: 11/743,571

(22) Filed: May 2, 2007

Related U.S. Application Data
(60) Provisional application No. 60/796,937, filed on May 3, 2006.

(51) Int. Cl. G01N 33/20; C07C 245/08

(52) U.S. Cl. 436/80; 534/848

(57) ABSTRACT

The invention relates to methods of determining calcium ions in a sample, wherein the ions are contacted with a compound having chromophoric moiety and an ionophoric moiety, where the ionophoric moiety interacts with the calcium ions present in the sample, resulting in the chromophoric moiety changing its radiation absorption properties in the ultraviolet and visible regions of the spectrum. For example, a change in an intensity of an absorption maximum is measured and the ion concentration is determined accordingly.

### BACKGROUND OF THE INVENTION

The measurement of ionized calcium in blood or serum is of importance in clinical diagnosis for many diseases such as hypoparathyroidism, tumor metastasis, renal failure and etc. Traditionally, it was determined in plasma or serum using ion-selective electrodes (see C. A. Burtis and E. R. Ashwood, Tietz Textbook of Clinical Chemistry, 3^{rd}, Saunders, Philadelphia, 1999). However, with the rapid growth of near-patient devices used at the hospital bedside, there is increasing demand for portable systems utilizing small disposable sensors capable of whole-blood measurements. Consequently, the development of practical and inexpensive optical sensors and systems for the clinical determination of ionized calcium in whole blood remains an important area of research (see J. P. Desvergne, A. W. Czarnik, Eds., Chemosensors of Ion and Molecule Recognition, NATO ASI Series, Kluwer Academic Publishers, Dordrecht, The Netherlands, 1996 and O. S. Wolfbeis, Fiber Optic Chemical Sensors and Biosensors, Vol. II; Ed., CRC Press, Boca Raton, 1991).

Many optical sensing schemes for calcium involving multiple types of molecules have been described, such as ion-exchange between the measured ion and a proton measured with a lipophilic pH-sensitive indicator dye. (see W. E. Morf, et. al., Pure Appl. Chem. 1989, 61, 1613-1620; and W. E. Molf, et. al. Anal. Chem. 1990, 62, 738-742), or interaction of a potential-sensitive dye with a neutral ion carrier. (see O. S Wolfbeis, et. al., Anal. Chim. Acta, 1987, 198, 1-7) and fluorescence calcium indicators (see R. Y. Tsien, Biochemistry, 1980, 19, 2396-2404). Most measuring methods based on neutral ion carriers suffer from inherent pH-dependencies (if based on pH indicators or proton exchange), and/or instabilities associated with leaching of critical components (if based on an ionophore to extract the desired cation into a lipophilic polymer membrane). Therefore, fluorescence calcium indicators with a covalently immobilizable group become the only choice for a practical useful candidate for such an application.

A fluorescence calcium indicator for intra-cellular calcium was first reported by R. Tsien (see R. Y. Tsien, Biochemistry, 1980, 19, 2396-2404) and has drawn a lot of attention since then. A number of publications have appeared in the following two decades. (see R. P. Haugland, Handbook of Fluorescent Probes and Research Products, 9^{th} Edition, 2001, 767-826). Despite of the difference of fluorophores, almost all of them are based on BAPTA [1,2-Bis-(o-AminoPhenoxy)ethane-N,N,N′N′-tetraacetic acid) aromatized from EGTA [Ethylenen Glycol bis(β-aminoethyl)-N,N,N′N′-Tetraacetic acid] with a dissociation constant in the range of micromolar. For determination of extra-cellular ionoized calcium whose concentration lies in milli-molar range, these fluorescent calcium indicators bind calcium about thousands times too tightly. One way to weaken the binding strength is to put an electron-withdrawing groups such as nitro, halogens on one of the aromatic ring. Those electron-withdrawing groups do suppress the bindings, but also create some other problems such as fluorescence quenching, and synthetic difficulties. Another way to weaken to binding is to use only half of binding unit of BEPTA, namely o-Anisidine-N,N-diacetic acid, which apparently was first reported by Irvine and Da Silva (see H. Irvine and J. J. R. F. Da Silva, J. Chem. Soc., 1963, 3308-3320), which gave a binding strength in the millimolar range with adequate selectivity against magnesium in extra-cellular application. However, the ionophore decomposed in the aqueous solution at pH 7.40 after 1 month storage at room temperature. A similar instability of BAPTA was also reported, especially for acidic form of BAPTA (see R. Y. Tsien et. al., U.S. Pat. No. 4,603,209). We found that the decomposition resulted from de-alkylation of acetic acid from aniline. The instability of this type of ionophore with an acetic acid linked directly to the aromatic nitrogen preludes their application to our system, in which the wet storage stability is an essential requirement.

U.S. Pat. No. 6,171,866 reports a calcium ionophore, which has π-electron conjugated nitrogen and was coupled to a fluorophore to make luminophore-ionophore sensors where the respective ions are detected by measuring luminescence emission. This ionophore has been proven to be very selective in determination of calcium in presence of magnesium in whole blood (see J. Tusa, et. al. J. Mater. Chem. vol. 15, 2005, 2640-2647), thereby showing that the ionophores are effective at physiological conditions.

By coupling to a chromophoric moiety, these ionophores can be converted into colorimetric sensors. The chromophoric moieties can be a nitro-substituted styryl or phenylazo, substituted thiazolevinyl or thiazoleazo, substituted naphthothiazolevinyl or naphthothiazoleazo, substituted naphthylvinyl or naphthylazo, substituted quinolinovinyl or quinolinoazo and their quarternized salts. To date, there has been no systematic investigation of these types of colorimetric reagents.

The present invention provides chromoionophores that are water soluble and can be reliably used for detection of ions in samples that absorb at wavelengths longer than about 400 nm. Examples of such samples are biological fluids.

The chromoionophores of this invention will absorb visible light (about 400 nm or greater) with reasonable extinction coefficient, thus avoiding those practical problems associated with variable background absorption from optical components, cuvette polymer materials, and biological samples.

For the chromoionophores of the present invention, the amount of ion present is determined by measuring changes in the intensity of at least one absorption maximum of the chromoionophore upon contacting the chromoionophore with an ion. The measurements are done by using standard centralized instruments, such as ultraviolet-visible spectrometers. A calibration curve for an ion is generated from a series of empirically determined absorption spectra. A calibration curve is useful for at-once determining the concentration of ion in a sample from the measured absorbance.

### SUMMARY OF THE INVENTION

The present invention relates to novel chromoionophores, comprising a chromophoric moiety and an ionophoric moiety. The invention further relates to a method of determining calcium ions in a sample, wherein the ions are contacted with a compound having chromophoric moiety and an ionophoric moiety, where the ionophoric moiety interacts with the calcium ions present in the sample, resulting in the chromophoric moiety changing its radiation absorption properties in the ultraviolet and visible regions of the spectrum. In one embodiment, a change in an intensity of an absorption maximum is measured and the ion concentration is determined accordingly.

In one embodiment, the chromoionophores of the invention comprise an ionophore having one or more chelating moieties that is capable of selectively binding calcium ions and a chromophore having a plurality of conjugated unsaturated bonds. The chromoionophore exhibits at least one absorption maximum having a wavelength in the visible region having a first intensity and wherein the absorption maximum has a second intensity that is different from the first intensity by an amount that is proportional to the concentration of calcium ion present in a mixture comprising calcium ions and the chromoionophore.

In other embodiments, chromoionophores of the invention are compounds having the Formula (I) 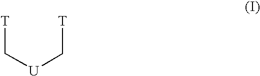
wherein T is COOH or carboxylate, and U is selected from the group consisting of three groups as described herein.

The group U can be a group having the general Formula II 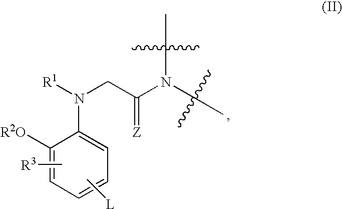
where R¹ is selected from the group consisting of (C₁-C₈) alkyl and aryl(C₁-C₈) alkyl, wherein any alkyl portion is optionally interrupted by one or more oxygens; R² is (C₁-C₈) alkyl optionally interrupted by one or more oxygens; R³ is selected from the group consisting of hydrogen, (C₁-C₈) alkoxy, halogen, —NO, and —NO₂.

Z is selected from the group consisting of H₂ and O, and L is a chromophoric moiety;

The group U can be a group having the general Formula III 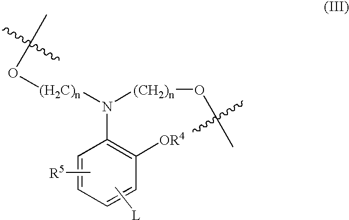
wherein n is 2 or 3; R⁴ is (C₁-C₈) alkyl optionally interrupted by one or more oxygens; R⁵ is selected from the group consisting of hydrogen, (C₁-C₈) alkoxy, halogen, —NO, and —NO₂; and L is a chromophoric moiety.

The group U can be a group having the Formula IV 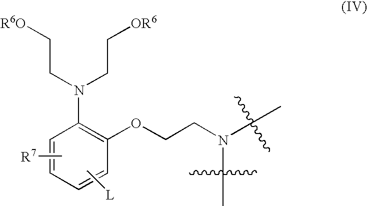
wherein R⁶ is selected from the group consisting of (C₁-C₃) alkyl and phenyl; R⁷ is selected from the group consisting of hydrogen, (C₁-C₈) alkoxy, halogen, —NO and —NO₂; and L is a chromophoric moiety.

The invention further provides a method of determining the concentration of calcium ions in a sample comprising

(a) measuring the intensity of at least one absorption maximum of a solution of a chromoionophore sensitive to the presence of calcium ions in solution to obtain a first intensity; wherein the concentration of the chromoionophore in solution is known; and

wherein said at least one absorption maximum has a wavelength in the visible region;

(b) contacting the solution of the chromoionophore with the sample; whereby the first intensity changes;

(c) measuring the intensity of at least one absorption maximum to obtain a second intensity;

(d) deriving the concentration of calcium ion in the sample based, in part, on the difference between the first and second intensities.

In one embodiment, at least one absorption maximum occurs at a wavelength that is in the visible region.

In another embodiment, the difference between the first and second intensities results in a colorimetric change in the solution sample comprising the chromoionophore and calcium ions.

In another embodiment, at least one absorption maximum occurs at a wavelength of about 400 nm or greater.

In another embodiment, at least one absorption maximum occurs at a wavelength between about 400 nm and about 800 nm.

### BRIEF DESCRIPTION OF THE DRAWINGS

FIG. 1 is an illustration of a synthetic pathway to a calcium colorimetric indicator.

FIG. 2 is a graph illustrating the absorbance of a calcium colorimetric indicator in accordance with the invention versus calcium concentration aqueous sample.

### DETAILED DESCRIPTION OF THE INVENTION

As used herein, the terms have the following meanings:

The term “alkyl” as used herein refers to a straight or branched chain, saturated hydrocarbon having the indicated number of carbon atoms. For example, (C₁-C₆) alkyl is meant to include, but is not limited to methyl, ethyl, propyl, isopropyl, butyl, sec-butyl, tert-butyl, pentyl, isopentyl, neopentyl, hexyl, isohexyl, and neohexyl. An alkyl group can be unsubstituted or optionally substituted with one or more substituents.

The term “alkylene” refers to a divalent alkyl group (e.g., an alkyl group attached to two other moieties, typically as a linking group). Examples of a (C₁-C₇) alkylene include —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, and —CH₂CH₂CH₂CH₂CH₂CH₂CH₂—, as well as branched versions thereof. An alkylene group can be unsubstituted or optionally substituted with one or more substituents.

The term “alkoxy” as used herein refers to an —O-alkyl group having the indicated number of carbon atoms. For example, a (C₁-C₆) alkoxy group includes —O-methyl, —O-ethyl, —O-propyl, —O-isopropyl, —O-butyl, —O-sec-butyl, —O-tert-butyl, —O-pentyl, —O-isopentyl, —O-neopentyl, —O-hexyl, —O-isohexyl, and —O-neohexyl.

The term “alkenyl” as used herein refers to a straight or branched chain unsaturated hydrocarbon having the indicated number of carbon atoms and at least one double bond. Examples of a (C₂-C₈) alkenyl group include, but are not limited to, ethylene, propylene, 1-butylene, 2-butylene, isobutylene, sec-butylene, 1-pentene, 2-pentene, isopentene, 1-hexene, 2-hexene, 3-hexene, isohexene, 1-heptene, 2-heptene, 3-heptene, isoheptene, 1-octene, 2-octene, 3-octene, 4-octene, and isooctene. An alkenyl group can be unsubstituted or optionally substituted with one or more substituents.

The term “Ar” as used herein refers to an aromatic or heteroaromatic moiety. An “aromatic” moiety refers to a 6- to 14-membered monocyclic, bicyclic or tricyclic aromatic hydrocarbon ring system. Examples of an aromatic group include phenyl and naphthyl. An aromatic group can be unsubstituted or optionally substituted with one or more substituents. The term “heteroaromatic” as used herein refers to an aromatic heterocycle ring of 5 to 14 members and having at least one heteroatom selected from nitrogen, oxygen and sulfur, and containing at least 1 carbon atom, including monocyclic, bicyclic, and tricyclic ring systems. Representative heteroaromatics are triazolyl, tetrazolyl, oxadiazolyl, pyridyl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, quinazolinyl, pyrimidyl, azepinyl, oxepinyl, naphthothiazolyl, quinoxalinyl. A heteroaromatic group can be unsubstituted or optionally substituted with one or more substituents.

The term “halogen” as used herein refers to —F, —Cl, —Br or —I.

As used herein, the term “heteroatom” is meant to include oxygen (O), nitrogen (N), and sulfur (S).

The term “chromoionophore” as used herein refers to a compound comprising at least one ionophore and at least one chromophore.

The following abbreviations are used herein and have the indicated definitions: LAH is lithium aluminum hydride; DMF is dimethylformamide; NMR is nuclear magnetic resonance; THF is tetrahydrofuran.

### Compounds of the Invention

The present invention provides compounds of Formula (I) referred to as “chromoionophores” 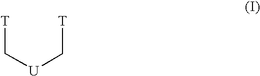
where T and U are as defined above.

In one embodiment, the chromophoric moiety L is selected from the group consisting of —NO₂, Formula (V) and (VI), 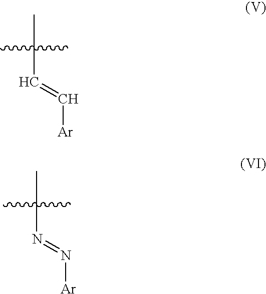
wherein, Ar is a (C₆-C₁₀) aromatic moiety or a (C₅-C₁₄) heteroaromatic moiety containing one or more heteroatoms selected from N, O, and S, and wherein Ar is substituted with one or more substituents selected from the group consisting of hydrogen, —NO₂, —NO, —CN, (C₁-C₈) straight chain or branched alkyl, (C₂-C₈) alkenyl, halogen, —SO₃H, —W—COOH, —W—N(R⁸)₃, —C(O)OR⁸, —C(O)R⁸; W is (C₁-C₈) alkylene; and R⁸ is selected from the group consisting of hydrogen and (C₁-C₈) straight chain or branched alkyl.

In another embodiment, Ar is selected from the group consisting of Formula (VII), (VIII), (IX), and (X)

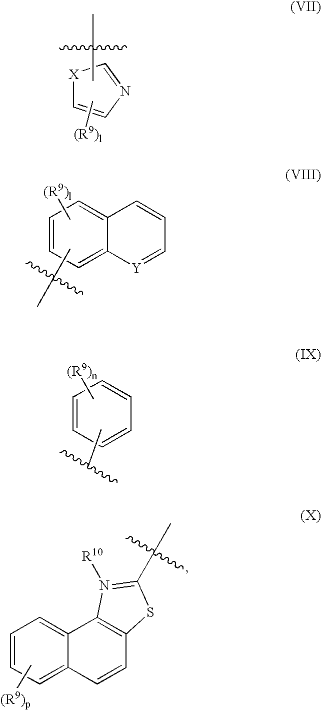

wherein X is O or S, and Y is N or C.

R⁹, at each occurrence, is independently selected from the group consisting of hydrogen, —NO₂, —NO, —CN, C₁-C₈ straight chain or branched alkyl, (C₂-C₈) alkenyl, halogen, —SO₃H, -Q-COOH, -Q-N(R¹¹)₃, —C(O)OR¹¹, —C(O)R¹¹.

R¹⁰ is -Q-SO₃⁻ or -Q-COO⁻.

Q is (C₁-C₈) alkylene.

R¹¹ is selected from the group consisting of hydrogen and (C₁-C₈) straight chain or branched alkyl.

Variable l is an integer selected from 1 to 3; m is an integer selected from 1 to 7; n is an integer selected from 1 to 5; and p is an integer selected from 1 to 6.

In one embodiment, U is the group of Formula (II).

In another embodiment, U is the group of Formula (III).

In still another embodiment, U is the group of Formula (IV).

Specific examples of compounds of Formula I are provided below:

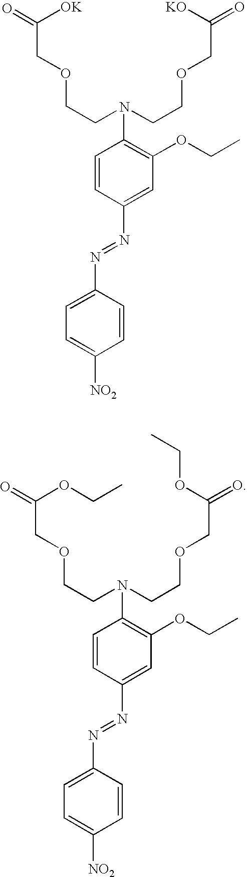

In one embodiment, the invention provides for a method of determining calcium ions in a sample comprising a chromoionophore and calcium ions, where the chromoionophore is a compound of the general Formula (I) 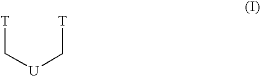
wherein T is COOH or carboxylate, and U is selected from the group consisting of three groups as described herein.

The group U can be a group having the general Formula II 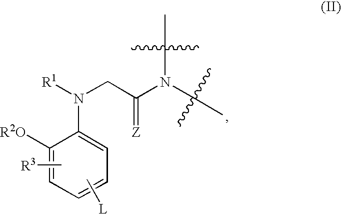
where R¹ is selected from the group consisting of (C₁-C₈) alkyl and aryl(C₁-C₈) alkyl, wherein any alkyl portion is optionally interrupted by one or more oxygens; R² is (C₁-C₈) alkyl optionally interrupted by one or more oxygens; R is selected from the group consisting of hydrogen, (C₁-C₈) alkoxy, halogen, —NO, and —NO₂.

Z is selected from the group consisting of H₂ and O, and L is a chromophoric moiety;

The group U can be a group having the general Formula III 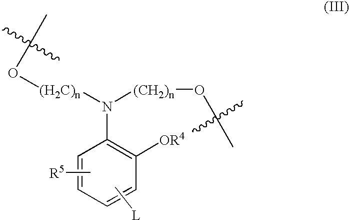
wherein n is 2 or 3; R⁴ is (C₁-C₈) alkyl optionally interrupted by one or more oxygens; R⁵ is selected from the group consisting of hydrogen, (C₁-C₈) alkoxy, halogen, —NO, and —NO₂; and L is a chromophoric moiety.

The group U can be a group having the Formula IV 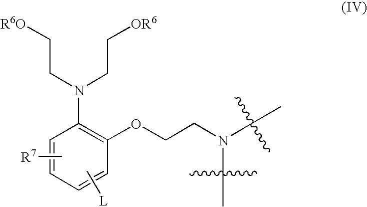
wherein R⁶ is selected from the group consisting of (C₁-C₃) alkyl and phenyl; R⁷ is selected from the group consisting of hydrogen, (C₁-C₈) alkoxy, halogen, —NO and —NO₂; and L is a chromophoric moiety.

The invention further provides methods of determining calcium ion in a sample comprising a chromoionophore according to Formula (I) and calcium ions, where the chromophoric moiety L is selected from the group consisting of —NO₂, Formula (V) and (VI), 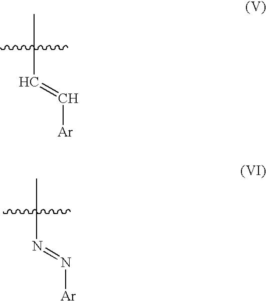
wherein, Ar is a (C₆-C₁₀) aromatic moiety or a (C₅-C₁₄) heteroaromatic moiety containing one or more heteroatoms selected from N, O, and S, and wherein Ar is substituted with one or more substituents selected from the group consisting of hydrogen, —NO₂, —NO, —CN, (C₁-C₈) straight chain or branched alkyl, (C₂-C₈) alkenyl, halogen, —SO₃H, —W—COOH, —W—N(R⁸)₃, —C(O)OR⁸, —C(O)R⁸; W is (C₁-C₈) alkylene; and R⁸ is selected from the group consisting of hydrogen and (C₁-C₈) straight chain or branched alkyl.

The invention further provides methods of determining calcium ion in a sample comprising a chromoionophore according to Formula (I) and calcium ions, where Ar is selected from the group consisting of Formula (VII), (VIII), (IX), and (X) 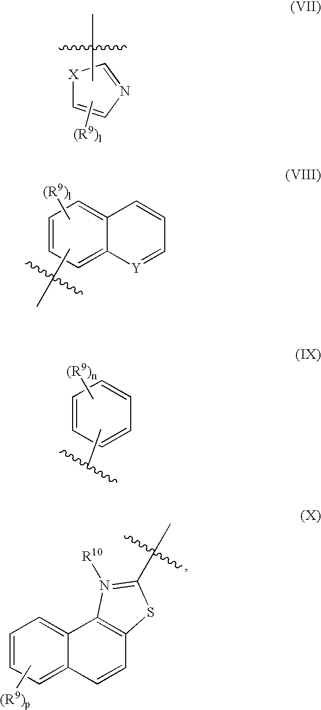
wherein X is O or S, and Y is N or C.

R⁹, at each occurrence, is independently selected from the group consisting of hydrogen, —NO₂, —NO, —CN, C₁-C₈ straight chain or branched alkyl, (C₂-C₈) alkenyl, halogen, —SO₃H, -Q-COOH, -Q-N(R¹¹)₃, —C(O)OR¹¹, —C(O)R¹¹.

R¹⁰ is -Q-SO₃⁻ or -Q-COO⁻.

Q is (C₁-C₈) alkylene.

R¹¹ is selected from the group consisting of hydrogen and (C₁-C₈) straight chain or branched alkyl.

Variable l is an integer selected from 1 to 3; m is an integer selected from 1 to 7; n is an integer selected from 1 to 5; and p is an integer selected from 1 to 6.

In one embodiment, the invention provides methods of determining calcium ion in a sample comprising a chromoionophore according to Formula (I) and calcium ions, where U is the group of Formula (II).

In another embodiment, the invention provides methods of determining calcium ion in a sample comprising a chromoionophore according to Formula (I) and calcium ions, where U is the group of Formula (III).

In still another embodiment, the invention provides methods of determining calcium ion in a sample comprising a chromoionophore according to Formula (I) and calcium ions, where U is the group of Formula (IV).

The invention further provides methods of determining calcium ion in a sample comprising a chromoionophore according to Formula (I) and calcium ions, where the sample is a biological fluid. Examples of biological fluids are whole blood, plasma, serum, and urine.

The invention further provides methods of determining calcium ion in a sample comprising a chromoionophore according to Formula (I) and calcium ions, where the sample has a pH of 6.5 or above.

Preparation of the Compounds of Formula (1)

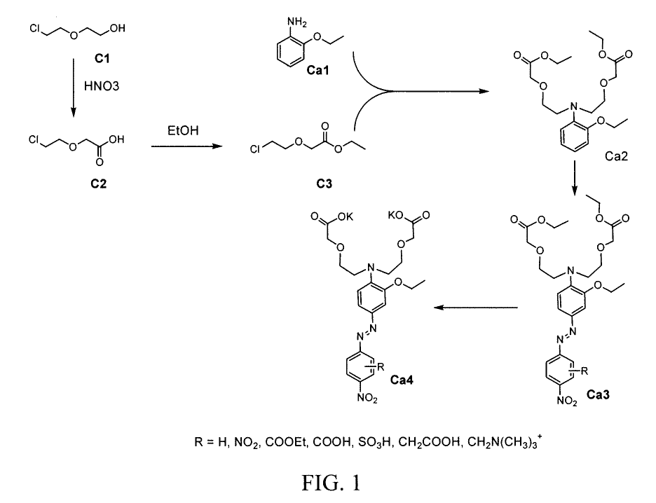

Those skilled in the art will recognize that there are a variety of methods available to synthesize molecules described herein. The synthesis of the chromoionophore (Ca4) from commercially available compounds is illustrated in FIG. 1. Chloroethoxyethanol (C1) was oxidized with HNO3 to give chloroethoxyacetic acid, which is esterfied in ethanol to get C3. o-Phenetidine (Ca1) was di-alkylated with C3 to give calcium ionophore Ca2, which was coupled with different diazoniums to afford chromoionophores (Ca3 and Ca4).

### EXAMPLE 1

Synthesis of C2. 118 mL (1.12 mol) 2-chloroethoxyethanol (C1) was added slowly into conc. HNO₃ (70%) (625 mL) at 55° C. within about 8 h period. The solution was stirred at RT for additional 18 h and heated in boiling water bath for 1 h. The solution was cooled, poured into icy water (500 mL). The diluted solution was extracted with CHCl₃ (5×1 L). All extractions were combined and dried over Na₂SO₄, Solvent was evaporated to afford 83.6 g oil. This oil was used directly for next esterification without further purification. ¹H NMR (300 MHz, CDCl₃): δ=3.72 (t, 2H, —CH₂Cl), 3.80 (t, 2H, CH₂O), 4.25 (s, 2H, OCH₂COOH), 10.40 (s,br. 1H, COOH).

### EXAMPLE 2

Synthesis of C3. A solution of 81.6 g (590 mmol) C2 in 575 mL absolute ethanol containing 1 mL conc. H₂SO₄ was heated under reflux for 18 h. Most of ethanol was evaporated. The residue was dissolved in CHCl₃ (600 mL) and washed with sat. NaHCO₃ (3×600 mL), dried over Na₂SO₄. The solvent was evaporated to afford 73.5 g clear oil. ¹H NMR (300 MHz, CDCl₃): δ=1.25 (t, 3H, —CH₃), δ=3.72 (t, 2H, —CH₂Cl), 3.80 (t, 2H, CH₂O), 4.15 (s, 2H, OCH₂COOH), 4.20 (q, 2H, COOCH₂CH₃). Anal. Calcd. for C₆H₁₁ClO₃: C, 43.26; H, 6.65. Found: C, 43.01; H, 6.81.

### EXAMPLE 3

Synthesis of Ca2. A suspension of 0.68 g (5 mmol) o-phenetidine (Ca1), 2.50 g (15 mmol) ethyl chloroethoxyacetate (C3), 2.07 g (15 mmol) K₂CO₃, 1.25 g (7.5 mmol) KI in 3 mL DMF was heated at 95° C. for 20 h. The mixture was cooled and diluted with 80 mL water/80 mL CHCl₃. The organic phase was washed with 80 mL sat. NaCl, dried over Na₂SO₄. Solvent was evaporated to give 2.05 g crude oil. This oil was purified with a plug packed with 5 g silica gel 100 using cyclohexane/CHCl₃ as eluent to remove front impurities, then using CHCl₃/ethyl acetate (4/1,v/v) to afford 0.84 g light yellow oil. H¹NMR (CDCl₃) δ (ppm) 1.22 (t,6H), 1.42 (t,3H), 3.42(t,3H), 3.62(t,3H), 4.02(q,2H),4.05(s,4H), 4.20(q,4H).6.80-7.05(m,4H).

### EXAMPLE 4

Synthesis of Ca3: To a suspension of Ca2 (2.0 g, 5 mmol) and 0.82 g (10 mmol) sodium acetate in 25 mL acetic acid was added 2.36 g (10 mmol) 4-nitrophenyldiazonium tetrafluoroborate. The suspension was stirred at room temperature for 18 hours and then poured into 500 mL crushed ice. The supernatant was decanted and the remain was washed with 3×100 mL water. The crude product was purified with silica gel with cyclohexane and chloroform as eluent, afforded 1.80 g dark brown gum. H¹NMR (CDCl₃) δ (ppm) 1.25 (t,6H), 1.44 (t,3H), 3.42(t,4H), 3.62(t,4H), 4.05(q,2H), 4.08(s,4H), 4.25(q,4H).7.08-7.35(m,3H), 8.23 (d,2H), 8.45 (d, 2H).

### EXAMPLE 5

Synthesis of Ca4. To a solution of Ca3 (1.80, 3.3 mmol) in 50 mL tetrahydrofuran and methanol was added 25 mL 1.0 N KOH in 25 mL water. The resulting solution was heated under reflux for 4 h. After cooling, the solvent was evaporated and the residue was dissolved in 400 mL water, washed with 400 mL CH₃Cl to remove some water insoluble impurities. The aqueous solution was acidified with acetic acid to bring pH down to about 2, sat for 2 h. The resulting precipitate was filtered, washed with 2×50 mL water, dried at room temperature for 18 h to give 0.58 g black powder. H¹NMR (DMSO-D6) δ (ppm) 1.40 (t,3H), 3.38(t,4H), 3.60(t,4H), 4.01(q,2H), 4.12(s,4H), 7.12-7.45(m,3H), 8.20 (d, 2H), 8.41 (d, 2H).

### EXAMPLE 6

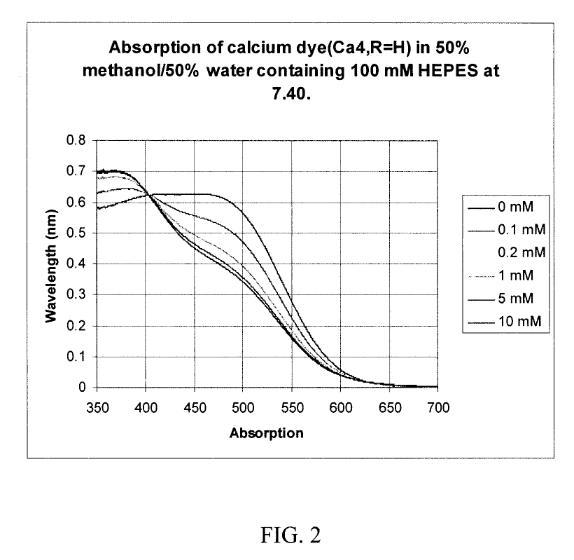

Method of Determining Calcium Ions: Solvents and reagents are purchased from Aldrich (Milwaukee, Wis.) and used without further purification. Analytical grade buffer and inorganic salts are purchased from either Fluka AG (Buchs, Switzerland) or Sigma Co. (St. Louis, Mo.). Absorption measurements are performed with a Shimadzu UV2101PC spectrophotometer equipped with a jacketed cuvette holder for controlling of temperature. Titration of a chromoinophore is carried out in the following manner: the dry powder of a chromoionophore is dissolved with buffer, deionized water or deionized water with organic co-solvent in a volumetric flask to make about 30 μM final solution, the required amount of solid salt is added and the solution's absorption spectrum is measured. The typical titration spectra are shown in FIG. 2.

What is claimed is:

1. A chromoionophore comprising
(a) an ionophore having one or more chelating moieties, wherein the ionophore is capable of selectively binding calcium ions, and
(b) a chromophore having a plurality of conjugated unsaturated bonds,
wherein the chromoionophore exhibits at least one absorption maximum having a wavelength in the visible region having a first intensity and wherein the absorption maximum has a second intensity that is different from the first intensity by an amount that is proportional to the concentration of calcium ion present in a mixture comprising calcium ions and the chromoionophore.

2. The chromoionophore according to claim 1, wherein at least one absorption maximum occurs at a wavelength of about 400 nm or greater.

3. The chromoionophore according to claim 1, wherein at least one absorption maximum occurs at a wavelength between about 400 nm and about 800 nm.

4. A compound selected from the group consisting of a compound having the general Formula (I): 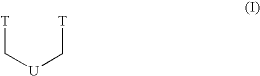
wherein
T is COOH or carboxylate;
U is selected from the group consisting of:
(a) a group having the general Formula II 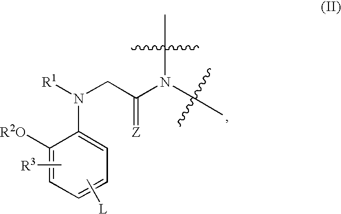
wherein
R¹ is selected from the group consisting of (C₁-C₈) alkyl and aryl(C₁-C₈) alkyl, wherein any alkyl portion is optionally interrupted by one or more oxygens;
R² is (C₁-C₈) alkyl optionally interrupted by one or more oxygens;
R³ is selected from the group consisting of hydrogen, (C₁-C₈) alkoxy, halogen, —NO, and —NO₂;
Z is selected from the group consisting of H₂ and O; and
L is a chromophoric moiety;
(b) a group having the general Formula III 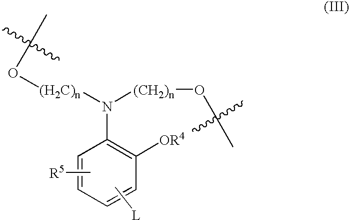
wherein n is 2 or 3;
R⁴ is (C₁-C₈) alkyl optionally interrupted by one or more oxygens;
R⁵is selected from the group consisting of hydrogen, (C₁-C₈) alkoxy, halogen, —NO, and —NO₂; and
L is a chromophoric moiety; and
(c) a group having the Formula IV 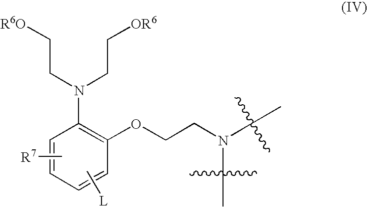
wherein R⁶ is selected from the group consisting of (C₁-C₃) alkyl and phenyl;
R⁷ is selected from the group consisting of hydrogen, (C₁-C₈) alkoxy, halogen, —NO and —NO₂; and
L is a chromophoric moiety.

5. The chromoionophore according to claim 4, wherein the chromophoric moiety L is selected from the group consisting of —NO₂, Formula (V) and (VI), 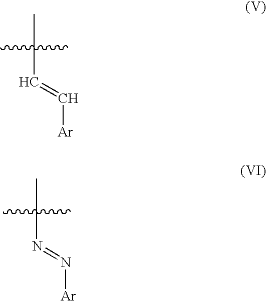
wherein,
Ar is a (C₆-C₁₀) aromatic moiety or a (C₅-C₁₄) heteroaromatic moiety containing one or more heteroatoms selected from N, O, and S, and wherein Ar is substituted with one or more substituents selected from the group consisting of hydrogen, —NO₂, —NO, —CN, (C₁-C₈) straight chain or branched alkyl, (C₂-C₈) alkenyl, halogen, —SO₃H, —W—COOH, —W—N(R⁸)₃, —C(O)OR⁸, and —C(O)R⁸;
W is (C₁-C₈) alkylene; and
R⁸ is selected from the group consisting of hydrogen and (C₁-C₈) straight chain and branched alkyl.

6. The chromoionophore according to claim 5, wherein Ar is selected from the group consisting of Formula (VII), (VIII), (IX), and (X) 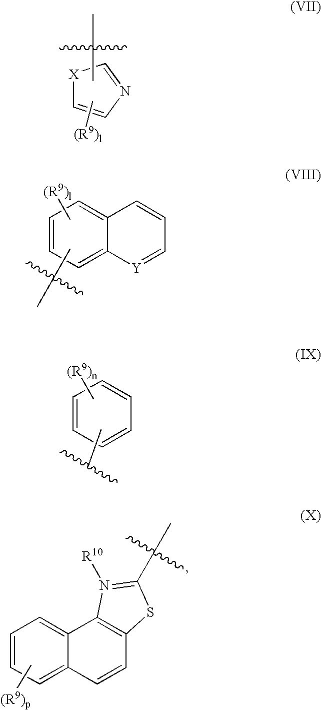
wherein
X is O or S;
Y is N or C;
R⁹, at each occurrence, is independently selected from the group consisting of hydrogen, —NO₂, —NO, —CN, C₁-C₈ straight chain or branched alkyl, (C₂-C₈) alkenyl, halogen, —SO₃H, -Q-COOH, -Q-N(R¹¹)₃, —C(O)OR¹¹, and —C(O)R¹¹;
R¹⁰ is -Q-SO₃⁻ or -Q-COO⁻;
Q is (C₁-C₈) alkylene;
R¹¹ is selected from the group consisting of hydrogen and (C₁-C₈) straight chain or branched alkyl;
l is an integer selected from 1 to 3;
m is an integer selected from 1 to 7;
n is an integer selected from 1 to 5; and
p is an integer selected from 1 to 6.

7. The chromoionophore according to claim 4, wherein U is the group of Formula (II).

8. The chromoionophore according to claim 4, wherein U is the group of Formula (III).

9. The chromoionophore according to claim 4, wherein U is the group of Formula (IV).

10. The chromoionophore according to claim 1, wherein the chromoionophore is selected from the group consisting of

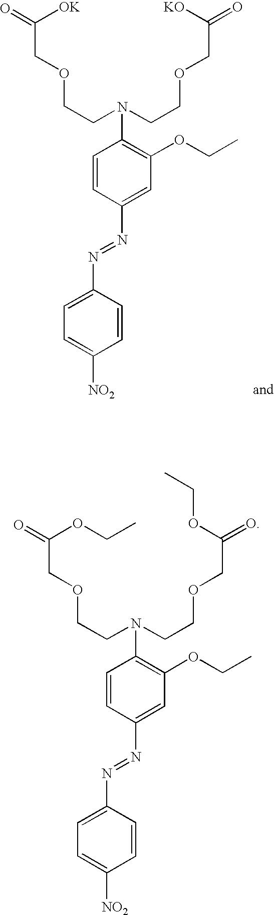

11. A method of determining the concentration of calcium ions in a sample comprising
(a) measuring the intensity of at least one absorption maximum of a solution of a chromoionophore sensitive to the presence of calcium ions in solution to obtain a first intensity; wherein the concentration of the chromoionophore in solution is known; and
wherein said at least one absorption maximum has a wavelength in the visible region;
(b) contacting the solution of the chromoionophore with the sample; whereby the first intensity changes;
(c) measuring the intensity of at least one absorption maximum to obtain a second intensity;
(d) deriving the concentration of calcium ion in the sample based, in part, on the difference between the first and second intensities.

12. The method according to claim 11, wherein at least one absorption maximum occurs at a wavelength of about 400 nm or greater.

13. The method according to claim 11, wherein at least one absorption maximum occurs at a wavelength between about 400 nm and about 800 nm.

14. The method according to claim 11, wherein the chromoionophore has the general Formula (I) 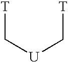
wherein
T is COOH or carboxylate;
U is selected from the group consisting of:
(a) a group having the general Formula II 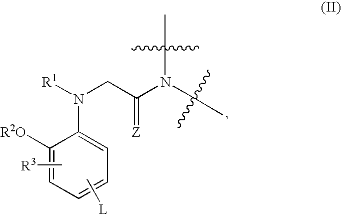
wherein
R¹ is selected from the group consisting of (C₁-C₈) alkyl and aryl(C₁-C₈) alkyl, wherein any alkyl portion is optionally interrupted by one or more oxygens;
R² is (C₁-C₈) alkyl optionally interrupted by one or more oxygens;
R³ is selected from the group consisting of hydrogen, (C₁-C₈) alkoxy, halogen, —NO, and —NO₂;
Z is selected from the group consisting of H₂ and O; and
L is a chromophoric moiety;
(b) a group having the general Formula III 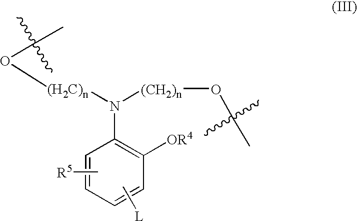
wherein n is 2 or 3;
R⁴ is (C₁-C₈) alkyl optionally interrupted by one or more oxygens;
R⁵ is selected from the group consisting of hydrogen, (C₁-C₈) alkoxy, halogen, —NO, and —NO₂; and
L is a chromophoric moiety; and
(c) a group having the Formula IV 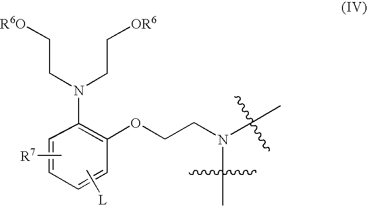
wherein R⁶ is selected from the group consisting of (C₁-C₃) alkyl and phenyl;
R⁷ is selected from the group consisting of hydrogen, (C₁-C₈) alkoxy, halogen, —NO and —NO₂; and
L is a chromophoric moiety.

15. The method according to claim 14, wherein the chromophoric moiety L is selected from the group consisting of —NO₂, Formula (V) and (VI), 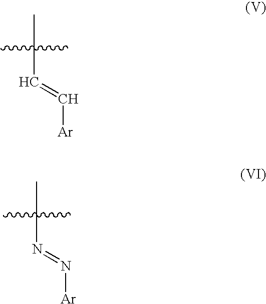
wherein,
Ar is a (C₆-C₁₀) aromatic moiety or a (C₅-C₁₄) heteroaromatic moiety containing one or more heteroatoms selected from N, O, and S, and wherein Ar is substituted with one or more substituents selected from the group consisting of hydrogen, —NO₂, —NO, —CN, (C₁-C₈) straight chain or branched alkyl, (C₂-C₈) alkenyl, halogen, —SO₃H, —W—COOH, —W—N(R⁸)₃, —C(O)OR⁸, and —C(O)R⁸;
W is (C₁-C₈) alkylene; and
R⁸ is selected from the group consisting of hydrogen and (C₁-C₈) straight chain or branched alkyl.

16. The method according to claim 15, wherein Ar is selected from the group consisting of Formula (VII), (VIII), (IX), and (X) 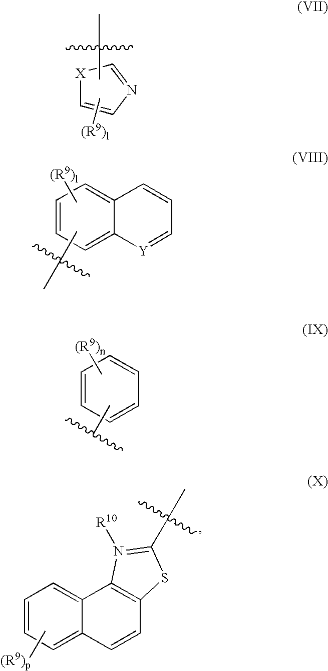
wherein
X is O or S;
Y is N or C;
R⁹, at each occurrence, is independently selected from the group consisting of hydrogen, —NO₂, —NO, —CN, C₁-C₈ straight chain or branched alkyl, (C₂-C₈) alkenyl, halogen, —SO₃H, -Q-COOH, -Q-N(R¹¹)₃, —C(O)OR¹¹, and —C(O)R¹¹;
R¹⁰ is -Q-SO₃⁻ or -Q-COO⁻;
Q is (C₁-C₈) alkylene;
R¹¹ is selected from the group consisting of hydrogen and (C₁-C₈) straight chain or branched alkyl;
l is an integer selected from 1 to 3;
m is an integer selected from 1 to 7;
n is an integer selected from 1 to 5; and
p is an integer selected from 1 to 6.

17. The method according to claim 11, wherein the chromoionophore is selected from the group consisting of

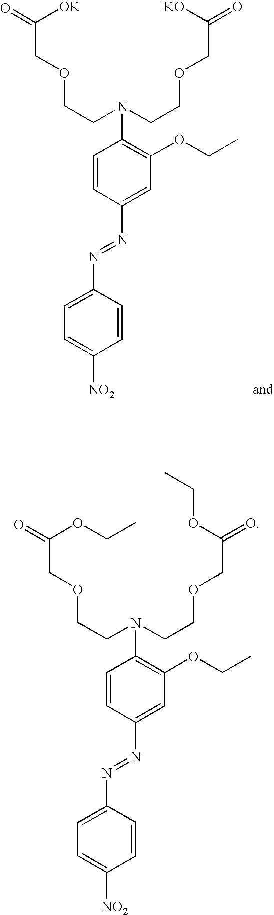

18. The method according to claim 11, wherein the sample is a biological fluid.

19. The method according to claim 18, wherein the biological fluid is selected from the group consisting of whole blood, plasma, serum, and urine.

20. The method according to claim 11, wherein the sample has a pH of 6.5 or above.
